(19) United States
(12) Plant Patent Application Publication
(10) Pub. No.: US 2006/0143752 P1
(43) Pub. Date: Jun. 29, 2006

(54) Chrysanthemum plant named 'Resomee White'

(50) CHRYSANTHEMUM MORIFOLIUM; CHRYSANTHEMUM

(75) Inventor: Mark Roland Boeder, The Hague (NL)

Correspondence Address: PARKHURST & WENDEL, L.L.P., 1421 PRINCE STREET, SUITE 210, ALEXANDRIA, VA, 22314-2805, US

(73) Assignee: Chrysanthemum Breeders Association N.V., Aalsmeer (NL)

(21) Appl. No.: 11/020,211

(22) Filed: Dec. 27, 2004

(51) Int. Cl. A01H 5/00

(52) U.S. Cl. PLT/288

(57) ABSTRACT

A Chrysanthemum plant named ‘Resomee White’ characterized by its large sized blooms with white ray-florets with a response time of 56 days.

### RELATED CULTIVARS

‘Resomee White’ is related to ‘Resomee’ (U.S. Plant Pat. No. 13,416), of which it is a sport.

### BACKGROUND OF THE INVENTION

Chrysanthemum morifolium L. ‘Resomee White’ is a product of a breeding and selection program which had the objective of finding color mutants of ‘Resomee’. The new plant of the present invention comprises a new and distinct cultivar of chrysanthemum plant that is a natural occurring sport of a parent chrysanthemum named ‘Resomee’. A comparison with parent chrysanthemum ‘Resomee’ is also made in this application. The new cultivar was discovered as a whole plant mutation in 2001 by Mark Roland Boeder in a controlled environment (greenhouse) in Rijsenhout Holland. The first act of asexual reproduction of ‘Resomee White’ was accomplished when vegetative cuttings were taken from the initial selection in 2001 in Rijsenhout, Holland.

### SUMMARY OF THE INVENTION

The present invention is a new and distinct variety of chrysanthemum bearing large sized blooms with white ray-florets.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

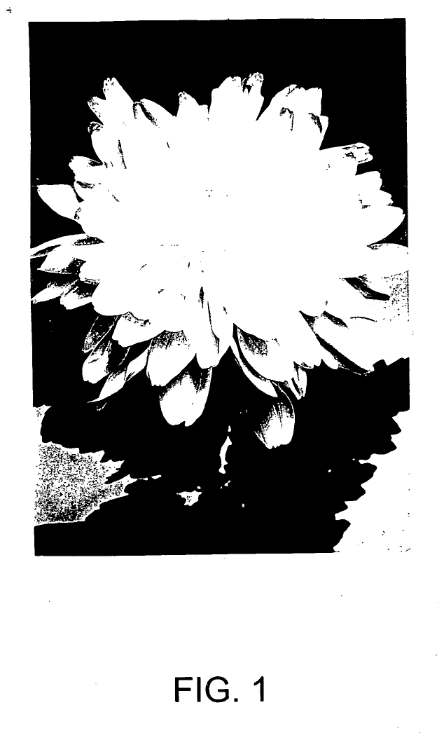

FIG. 2 shows the various stages of bloom of the new cultivar.

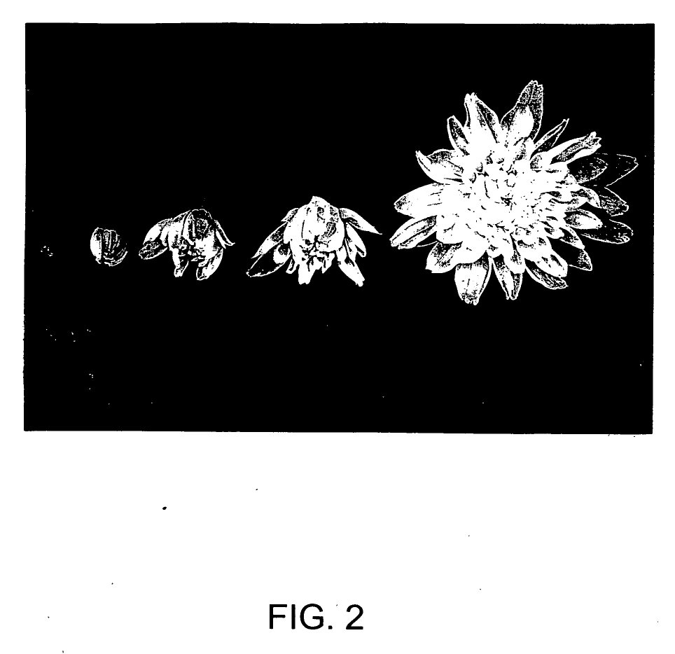

FIG. 3 shows the foliage of the new cultivar.

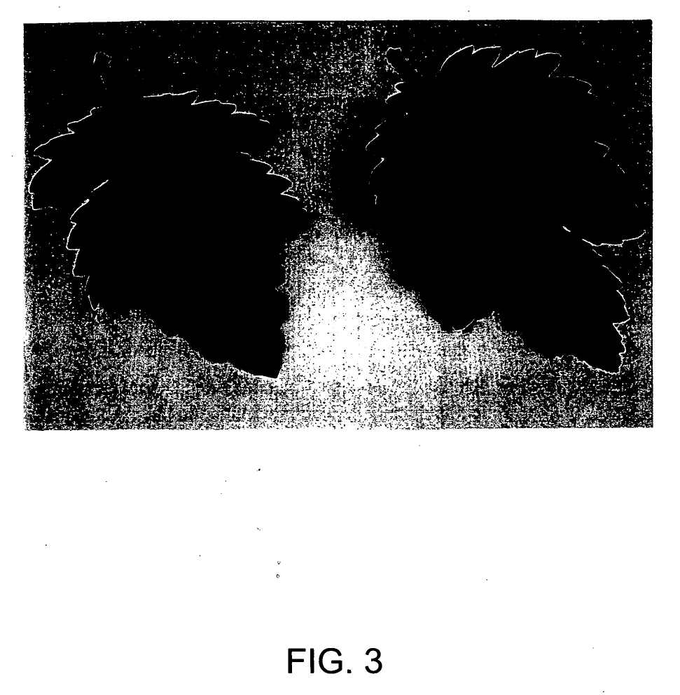

### DESCRIPTION OF THE INVENTION

This new variety of chrysanthemum is of the botanical classification Chrysanthemum morifolium L. The observations and measurements were gathered from plants grown in April/May in a greenhouse in Rijsenhout Holland in a photo-periodic controlled crop under conditions generally used in commercial practice. The greenhouse temperatures during this crop were at day-time between 18° C. and 25° C. and at night 20° C. After a long day period of 14 days the photo-periodic response time in this crop was 56 days. After the long day period to flowering growth retardants were applied 2 to 3 times in an average dose of 2.5 gram/liter water. No tests were done on disease or insect resistance or susceptibility. No tests were done on cold or drought resistance. This new variety produces large sized blooms with white ray-florets blooming on the plant for 3 weeks. This new variety of chrysanthemum has been found to retain its distinctive characteristics throughout successive propagations however the phenotype may vary significantly with variations in environment such as light intensity and temperature. To show the phenotype as described ‘Resomee White’ can be planted without assimilation lighting (high pressure sodium lamps) between week 1 and week 35 under greenhouse conditions in Holland. With assimilation light (minimum level 2500 lux) it can be planted year round under greenhouse conditions in Holland.

From the cultivars known to inventor the most similar existing cultivars in comparison to ‘Resomee White’ is ‘Resomee’ (U.S. Plant Pat. No. 13,416) and ‘Resolute’ (U.S. Plant Pat. No. 14,067). When ‘Resomee’, ‘Resomee White’ and ‘Resolute’ are being compared the following differences and similarities are noticed: The differences of ‘Resomee’, ‘Resomee White’, and ‘Resolute’ are (1) Color ray-florets (2) Color center of the bloom (3) Flowering response. (1) and (2) are characteristics for which ‘Resomee White’ has been selected out of ‘Resomee’. All other characteristics of ‘Resomee’ and ‘Resomee White’ are similar. (1) ‘Resomee White’ and ‘Resolute’ have both white colored ray florets, while the ray florets of ‘Resomee’ are pink. (2) The center of the bloom of ‘Resomee White’ is cream colored, while it is light green in ‘Resolute’. The flowers of ‘Resomee’ are all over pink. (3) The flowering response is 50 days for ‘Resolute’, and 56 days for the ‘Resomee’ family.

The following is a description of the plant and characteristics that distinguish ‘Resomee White’ as a new and distinct variety. The color designations are taken from the plant itself. Accordingly, any discrepancies between the color designations and the colors depicted in the photographs are due to photographic tolerances. The color chart used in this description is: The Royal Horticultural Society Color Chart, edition 1995.

TABLE 1
Botanical Description of cultivar ‘Resomee White’

| | |
|---|---|
| Bud | |
| Size | Large, cross-section 1 cm height |
| | 1 cm |
| Outside color | Yellow-green 147D-145D |
| Phyllaries | 2 rows, length 7 mm, width 3 mm |
| Phyllaries among disc florets | Not present |
| Bloom | |
| Type | Single decorative |
| Height | 3 cm |
| Size | large |
| Fully Expanded | 12 cm |
| Receptacle shape | Domed raised |
| Phyllary number | 25 |
| Phyllary shape | Lanceolate |
| Phyllary length and width | 0.8-1 cm; 0.3-0.4 cm |
| Phyllary apex | Acute |
| Phyllary base | Aneate |
| Phyllary margin | Margin |
| Phyllary color | Outside: Green 138A |
| | Inside: Green 139B |
| Performance on the plant | 3 weeks |
| Seeds (if crossed) | Produced in small quantities, oval |
| | shaped, grey-brown 199A, 2 mm |
| | in length. |
| Fragrance | Typical chrysanthemum |
| Color | |
| Center of the | Immature Yellow-white 158D |
| inflorescence | Mature Yellow-white 158D |
| Color of the ray- | Upper surface White 155A |
| florets | Lower surface White 155D |
| Tonality from Distance | A disbudded mum with large |
| | white flowers |
| Color of the upper surface of the | White 155D |
| ray florets after aging of the plant | |
| Disc florets | Not present |
| Ray florets | |
| Texture | Upper and under side smooth |
| Number | 180-190 |
| Cross-section | Flat to concave |
| Longitudinal axis of majority | Straight |
| Length of corolla tube | Medium; 1.0-1.4 cm |
| Ray-floret margin | Entire |
| Ray-floret length | 5-6 cm |
| Ray-floret width | 0.9-1.3 cm |
| Ratio length/width | high |
| Shape of tip | pointed |
| Reproductive Organs | |
| Stamen | Not present |
| Pollen | Not present |
| Styles (present in ray florets) | Thin |
| Style color | Yellow-green 145 D |
| Style Length | 3 mm |
| Stigma color | Yellow-green 145 D |
| Stigma Width | 1 mm |
| Ovaries | Enclosed in perianth |
| Plant | |
| Form | A disbudded mum meant for |
| | erect culture |
| Growth habit | Upright |
| Growth rate | high |
| Height | 100 cm |
| Width | 20 cm |
| Internode length | 2.5-3 cm |
| Stem Color | Green 138B |
| Stem Strength | Strong |
| Stem Brittleness | Absent |
| Stem Anthocyanin Coloration | Absent |
| Flowering Response(photo-periodic | 56 days |
| controlled crop, not natural season) | |
| Foliage | |
| Color immature stage | Upper side Green 139B-143B |
| | Under side Green 139C |
| Color mature stage | Upper side Green 139A-B |
| | Under side Green 137A-138A |
| Color midvein | Upper side Green 138D |
| | Under side Green 138B-C |
| Size | Large; length 9.5 cm, width 7 cm |
| Quantity (number per | 32-34 |
| single stem) | |
| Shape | Elliptic - circular |
| Texture upper side | Fleshy and glabrous |
| Texture under side | Pubescent |
| Venation arrangement | Palmate |
| Shape of the margin | Dentate |
| Shape of Base of Sinus | Acute |
| Between Lateral Lobes | |
| Margin of Sinus | Diverging |
| Between Lateral Lobes | |
| Shape of Base | Truncate |
| Apex | Mucronulate |
| Petiole length | 2-2.5 cm |
| Petiole color | Green 138D |

TABLE 2
Differences with the comparison Varieties

| | ‘Resomee White’ | ‘Resomee’ | ‘Resolute’ |
|---|---|---|---|
| Color ray florets | White 155A | Purple 75B | White 155A |
| Color center | Yellow-white | Purple 75B | Yellow-green |
| of bloom | 158D | | 145 A |
| Natural response | 56 days | 56 days | 50 days |

I claim:

1. A new and distinct variety of chrysanthemum plant as described and illustrated.
